(19) United States
(12) Patent Application Publication
(10) Pub. No.: US 2009/0240139 A1
(43) Pub. Date: Sep. 24, 2009

(54) Diffuse Optical Tomography System and Method of Use

(76) Inventor: Steven Yi, Vienna, VA (US)

Correspondence Address: STEVEN L. NICHOLS;RADER, FISHMAN & GRAUER PLLC, 10653 S. RIVER FRONT PARKWAY, SUITE 150, SOUTH JORDAN, UT, 84095, US

(21) Appl. No.: 12/050,793

(22) Filed: Mar. 18, 2008

(51) Int. Cl. A61B 6/03

(52) U.S. Cl. 600/425

(57) ABSTRACT

A diffuse optical tomography imaging system for in vivo non-contact imaging, includes an illumination source assembly for illuminating a specimen; a time-domain sensor assembly for capturing a time-domain response of the specimen to illumination from the illumination source assembly; a frequency-domain sensor assembly for capturing a frequency-domain response of the specimen to the illumination; and a three-dimensional (3D) imaging assembly for outputting an electronic (3D) model of the specimen. The system combines the 3D model and tomography data generated from the time-domain response and frequency-domain response for the specimen.

### BACKGROUND

Diffuse Optical Tomography (DOT) is optical imaging that is able to three-dimensionally resolve and quantify the bio-distribution of chromophores and fluorescence reporters through several millimeters to centimeters of tissue. With this ability comes the capacity to resolve various bio-markers and study disease evolution and the effects of treatment. DOT may also be used to measure physiological parameters, such as (1) oxygen saturation of hemoglobin and blood flow, based on intrinsic tissue contrast, (2) molecular tissue function, and (3) gene-expression based on extrinsically administered fluorescent probes and beans. DOT offers several potential advantages over existing radiological techniques, such as being non-invasive and non-ionizing. DOT may also aid cancer detection and treatment in cancer patients, such as breast cancer patients.

DOT imaging includes illuminating the tissue with a light source and measuring the light leaving the tissue with a sensor. A model of light propagation in the tissue is developed and parameterized in terms of the unknown scattering and/or absorption as a function of position in the tissue. Then, using the model together with the ensemble of images over all the sources, the DOT imaging system inverts the propagation model to recover the scatter and absorption parameters.

A DOT image is actually a quantified map of optical properties and can be used for quantitative three-dimensional imaging of intrinsic and extrinsic adsorption and scattering, as well as fluorophore lifetime and concentration in diffuse media such as tissue. These fundamental quantities can then be used to derive tissue oxy- and deoxy-hemoglobin concentrations, blood oxygen saturation, contract agent uptake, and organelle concentration. Such contrast mechanisms are important for practical applications such as the measurement of tissue metabolic activity, angiogenesis and permeability for cancer detection as well as characterizing molecular function.

A typical DOT system uses lasers so that specific chromophores are targeted and the forward model is calculated for the specific wavelengths used. Laser diodes have been customarily used as light sources since they produce adequate power and are stable and economical. Light is usually directed to and from tissue using fiber optic guides since this allows flexibility in the geometrical set-up used. For optical coupling, the fibers must be in contact with tissue or a matching fluid. Use of a matching fluid helps to eliminate reflections due to mismatches between indices of refraction between silica, air, and tissue.

Advanced DOT algorithms require good knowledge of the boundary geometry of the diffuse medium imaged in order to provide accurate forward models of light propagation within this medium. A forward model is a representation of the representative characteristics of the volume being studied. Currently, these boundary geometries are forced into simple, well known shapes such as cylinders, circles, or slabs. In addition to not accurately representing the shape of the specimen to be analyzed, the use of these shapes forces the specimen being analyzed to be physically coupled to the shape either directly or by the use of a matching fluid as discussed above.

In recent years several methods have been developed to model photon propagation through diffuse media with complex boundaries using Monte Carlo approaches, finite solutions of the diffusion or transport equation or more recently analytical methods based on the tangent-plane method. To fully exploit the advantages of these sophisticated algorithms, accurate 3D boundary geometry of the subject has to be extracted in practical, real-time and in-vivo manner.

Developing more accurate 3D imaging technology may have benefits in areas other than DOT such as facilitating 3D facial recognition, aiding reconstructive surgery using patient-specific 3D models for pre-operative planning and post-operative verification, creating and providing 3D models of dental structure, creating patient specific burn masks, and other applications where accurate 3D imaging may be desired.

### BRIEF DESCRIPTION OF THE DRAWINGS

The accompanying drawings illustrate various embodiments of the principles described herein and are a part of the specification. The illustrated embodiments are merely examples and do not limit the scope of the claims.

FIG. 1 is a diagram showing one illustrative embodiment of a diffuse optical tomography system, according to principles described herein.

FIG. 2 is a diagram showing one illustrative embodiment of a spectrum source assembly in combination with a three-dimensional (3D) sensor assembly, according to principles described herein.

FIG. 3 is a diagram showing one illustrative embodiment of positioning a 3D camera and a spectrum source assembly for light projection orientation.

FIG. 4 is a diagram showing one illustrative embodiment of a saw-toothed light pattern for rainbow 3D imaging.

FIG. 5 is a diagram showing one illustrative embodiment of a timing chart for synchronizing color patterns with capture cycles of a 3D camera.

FIG. 6 is a diagram showing one illustrative method of obtaining rainbow 3D data with a 3D imaging assembly.

FIG. 7 is a diagram showing one illustrative embodiment of a plurality of optical probes and detection fibers distributed on a supporting structure.

FIG. 8 is a diagram showing one illustrative embodiment of a frequency-domain source module.

FIG. 9 is a diagram showing one exemplary embodiment of a frequency-domain detection module.

Throughout the drawings, identical reference numbers designate similar, but not necessarily identical, elements.

### DETAILED DESCRIPTION

An improved diffuse optical tomography system for in vivo non-contact imaging includes an illumination source for illuminating a specimen, a spectrum source assembly for projecting a spectrum onto the specimen, at least one sensor configured to capture the response of the specimen to the illumination and to the projection of the spectrum. The improved diffuse optical tomography system rapidly captures three-dimensional boundary geometry of and the corresponding diffuse optical tomography measurements of a specimen.

As used herein and in the appended claims, the term “tomography data” will be used to refer to data that is produced, as described above, from Diffuse Optical Tomography or Fluorescence Molecular Tomography. Consequently, “tomography data” refers to data produced by passing light or other electromagnetic radiation through a tissue specimen and then recording and interpreting the resulting transmission and scattering of the light or other radiation by the specimen. As used herein, “specimen” shall be broadly understood to mean any volume or surface to be analyzed.

As used herein and in the appended claims, the term “pixel” shall be broadly understood to mean an individual element of a picture or the hardware used to produce or represent an individual picture element. As used herein and in the appended claims, the term “voxel” shall be broadly understood to mean any element of a three-dimensional or volumetric model, display or representation of a specimen or other three-dimensional body.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “an embodiment,” “an example” or similar language means that a particular feature, structure, or characteristic described in connection with the embodiment or example is included in at least that one embodiment, but not necessarily in other embodiments. The various instances of the phrase “in one embodiment” or similar phrases in various places in the specification are not necessarily all referring to the same embodiment.

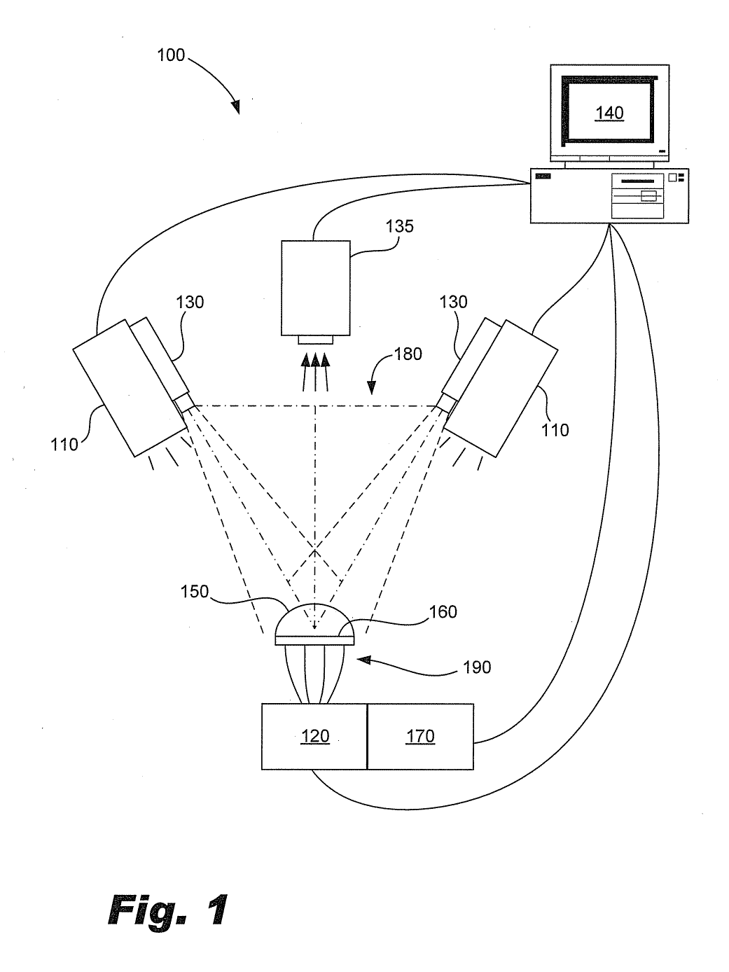

FIG. 1 illustrates an improved diffuse optical tomography (DOT) system (100) that comprises at least one spectrum source assembly (110), an illumination assembly (120), at least one three-dimensional (3D) imaging assembly (130), a time-domain sensor assembly (135), and a frequency-domain sensor assembly (170), all coupled to a processing device such as a computer (140). The system (100), with these components, is able to generate a 3D model or surface profile for the specimen (150) and then seamlessly and instantly associate tomography data with the 3D model. Consequently, the system provides a very accurate 3D model or framework in which tomography data is accurately placed. Each of the system components will be described in more detail below.

In the illustrated embodiment, the system (100) includes at least two spectrum source assemblies (110) and two corresponding 3D imaging assemblies (130). The spectrum source assemblies (110) project a spectrum or rainbow of visible or other light onto the surface of the specimen (150) resting on a supporting structure (160), such as a positioning plate. As will be described herein, the spectrum source assemblies (110) and the corresponding 3D imaging assemblies (130) produce the 3D model or surface profile for the specimen (150). In the illustrated example, two spectrum source assemblies and corresponding 3D imaging assemblies (130) are used to produce a complete 3D model of the surface of both sides of the specimen (150). For example, the spectrum source assembly (110) and corresponding 3D imaging assembly (130) on the right of FIG. 1 can observe and model portions of the specimen surface that are out of the line of sight of the other spectrum source assembly and corresponding 3D imaging assembly, and vice versa.

The illumination assembly (120) projects light onto and through the specimen (150). As shown in FIG. 1, the illumination assembly (120) is located on the opposite side of the specimen (150) from the time-domain sensor assembly (135). The illumination assembly (120), the time-domain sensor assembly (135), and the frequency-domain sensor assembly (170) are used to produce the desired tomography data regarding the specimen (150). As noted above, this tomography data may be registered and placed electronically within the 3D model of the specimen (150) produced by the 3D imaging assemblies (130). The time-domain sensor assembly (135) comprises a sensor, such as a near-infrared (NIR) camera. The time-domain sensor assembly (135) may also comprise filters for controlling the intensity or wavelength of the response captured by the sensor.

The data captured by the time-domain sensor assembly (135), the frequency-domain sensor assembly (170), and the 3D imaging assemblies (130) are output to the computer (140). The computer (140) processes this data to produce the desired tomography data regarding the specimen that is registered and placed electronically within a 3D model of the specimen (150). The computer (140) then displays the results on a display device attached to the computer, such as a monitor.

Any 3D imaging assembly and time-domain and frequency-domain sensor assemblies may be used to capture the specimen responses for generating the data transmitted to the computer. One example of a suitable 3D camera assembly (130) is one that is configured to be used with a spectrum source assembly (110), as in the embodiment shown in FIG. 1. However, other suitable 3D imaging assemblies include laser-scanning systems.

In the example shown in FIG. 1, the spectrum source assembly (110) is configured to project a spectrum of light of spatially varying wavelengths in the visible range onto the specimen (150). The response of the specimen to the applied light may be used to determine three-dimensional boundary conditions of the specimen. The three-dimensional boundary may be determined by utilizing triangulation. A triangle may be formed by the distance between the spectrum source assembly (110), the 3D imaging assembly (130), and a point on the specimen (150). The triangulation may also be determined by using a point on the specimen (150) and two 3D imaging assemblies (130), or between a point on the specimen (150) and a plurality of cameras in a single 3D imaging assembly (130). A system employing a plurality of 3D imaging assemblies (130) positioned at different locations may use a common baseline distance between the 3D imaging assemblies (130), which is a constant established during system configuration, and a distance between each 3D imaging assembly (130) and the point on the specimen (which distance is found by using the angles of the desired triangle) to ultimately determine the three-dimensional boundary conditions of the specimen. The distance between each camera in a 3D imaging assembly (130) and between each camera and the spectrum source may also be constants predetermined by system configuration. See, e.g., U.S. Pat. No. 6,147,760 to Geng, which is incorporated herein by reference in its entirety.

The illumination source assembly (120) is configured to apply light in, for example, the visible spectrum to the specimen (150) to generate tomography data. As indicated above, the response of the specimen (150) to the applied light from the illumination source assembly (120) may be used to determine internal characteristics of the specimen (150) such as the spectroscopic information about the biochemical structure of a tissue specimen, because the light from the illumination source assembly (120) may penetrate an outer surface of the specimen (150). The information about the biochemical structure, the tomography data, is obtained by capturing and processing the specimen's response to illumination from the illumination source assembly (120). This spectroscopic information may reveal physiological parameters (e.g., oxygen saturation of hemoglobin and blood flow) based on intrinsic tissue contrast, molecular tissue function, as well as gene-expression based on extrinsically administered fluorescent probes and/or beacons. The specimen's response may also be useful for detecting and treating cancer in breast cancer patients or others.

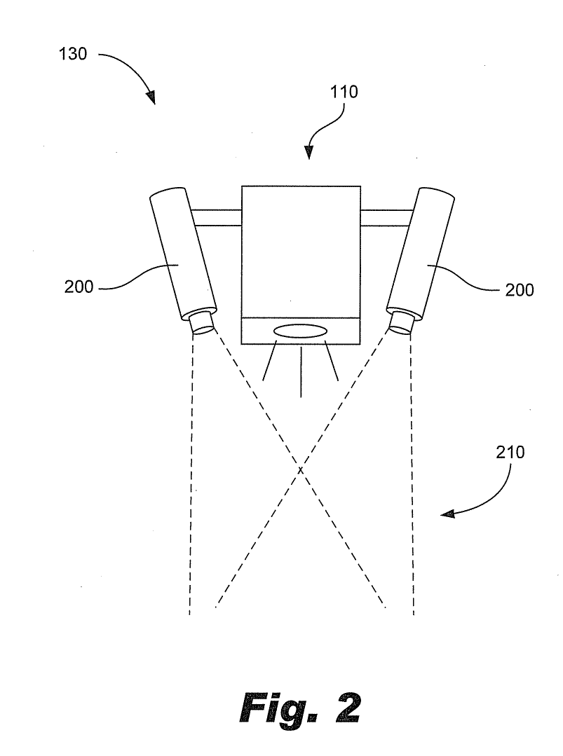

FIG. 2 illustrates a spectrum source assembly (110) in combination with a 3D imaging assembly (130) according to one illustrative embodiment. This spectrum source assembly (110) may comprise a projection light source. The projection light source may be a light emitting diode (LED) based pattern projector, though the projection light source may be any type of pattern projector. An LED-based pattern projector may be preferred because of its low power requirements and it may allow for construction of a smaller DOT imaging system than systems which use other types of pattern projectors or projection light sources. However, other projection light sources may be used which are capable of projecting a spectrum onto the specimen which may also meet desired size or power specifications.

The spectrum source assembly (110) may be capable of projecting a rainbow spectrum without any additional or exterior filters. One example of a spectrum source assembly with this capability is a digital light processing (DLP) projector, which uses millions of microscopic mirrors arranged in a rectangular array spaced less than 1 micron apart. The mirrors are capable of switching on and off thousands of times per second and are used to direct light towards, and away from, a dedicated pixel space. The duration of the on/off timing determines the level of gray seen in the pixel. DLP projectors are able to project red, green, and blue light sequentially at a very high speed. DLP projectors may generally be smaller in size, consume less power, have a longer life, cost less, and weigh less than other projection light sources capable of projecting a rainbow spectrum. In order to use a DLP projector for the current invention, the projector may need to be modified with customized trigger circuitry for synchronizing the cameras in the 3D imaging assembly (130) for shape acquisition.

Alternatively, the spectrum source assembly (110) may include a linear variable wavelength filter (LVWF, not shown). Light projected from the spectrum source assembly (110) through the LVWF falls onto the specimen (150) as a rainbow spectrum. The wavelength of the coated color of the LVWF in a specific location is linearly proportional to the displacement of the location from the LVWF's blue edge. Accordingly, the specific pixel characteristics at each point constrain the system, thereby providing accurate information about the three-dimensional location of the point. The spectrum source assembly (110) may comprise a plurality of variable density filters where the projection light source is a monochromic source, and the filters are sequentially placed in front of the monochromic source in order to effectively create a projection equivalent to a rainbow projection. The filters may also be color spectrum filters. Other suitable spectrum sources include laser-scanning systems.

The 3D imaging assembly (130) may comprise a plurality of video cameras (200) capable of capturing a response of the specimen to the rainbow projection emitted from the spectrum source assembly (110) over 180 degrees, preferably charge-coupled device (CCD) cameras. The fields of view (210) of each camera (200) in the 3D imaging assembly overlaps such that a specimen is visible by both cameras (200) and the cameras (200) are able to output a 3D image of the specimen. This allows a plurality of 3D camera assemblies (130) in the imaging system positioned at different locations to cover a full 360 degrees of the top portion of a specimen such that a complete hemispherical, three-dimensional rendering of the specimen may be adequately reconstructed in conjunction with the captured responses of the time-domain and frequency-domain sensor assemblies.

FIG. 3 is a diagram showing the positioning of a 3D imaging assembly (130) and a spectrum source assembly (110) for light projection orientation. In order to optimize the 3D output of the 3D imaging assembly (130), the light projection angle and the relative position between the camera(s) in the 3D imaging assembly (130) and the spectrum source assembly (110) may be carefully controlled during opto-mechanical design. The projector's principal ray (300) may meet with the camera's optical axis (310) at a center working distance (320). The spectrum source assembly (110) and the cameras in the 3D imaging assembly (130) may be tilted at an angle (330) in order to achieve this, or they may remain un-tilted. The angle (340) between the principal ray (300) and the optical axis (310), the angle (350) between the principal ray (300) and the normal (360) of the object plane (370), and the angle (380) between the optical axis (310) and the normal (360) may be minimized in order to achieve a better 3D image. If the cameras in the 3D imaging assembly (130) are un-tilted with respect to the normal (360), the angle (380) between the optical axis (310) and the normal (360) is zero. This makes the angle (340) between the principal ray (300) and the optical axis (310) equal to the angle (350) between the principal ray (300) and the normal (360), which may allow for simpler calculations. Reflective mirrors or other means of redirecting light may be used to bring the principal ray (300) closer to the optical axis (310), which may result in a higher quality three-dimensional image.

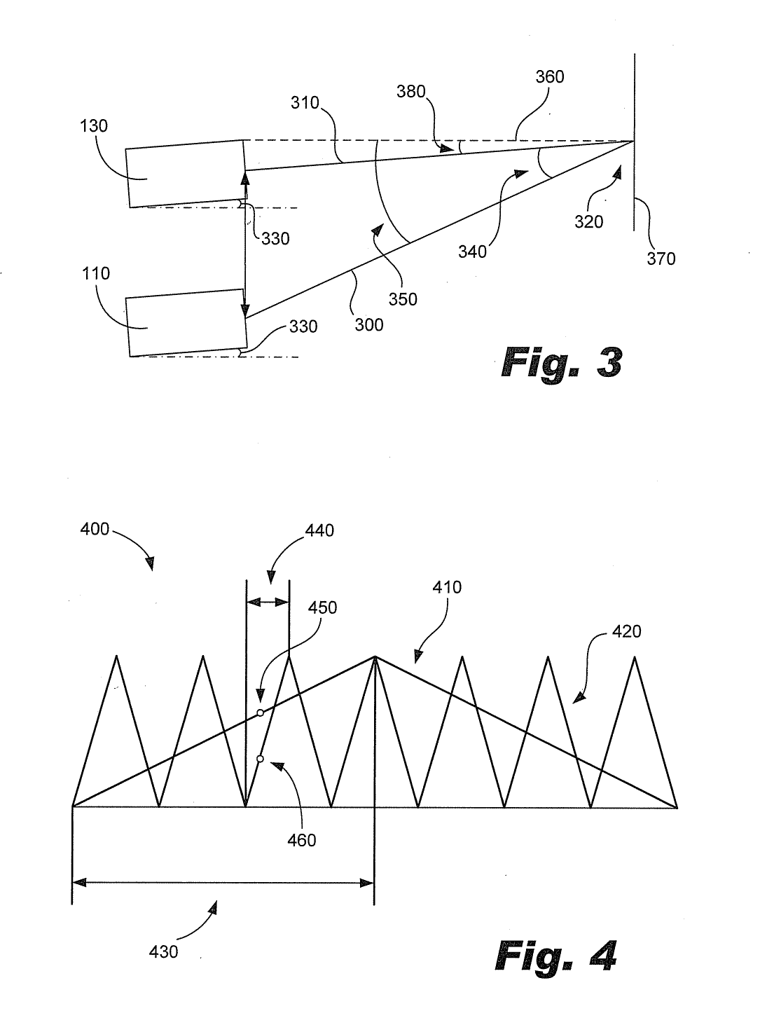

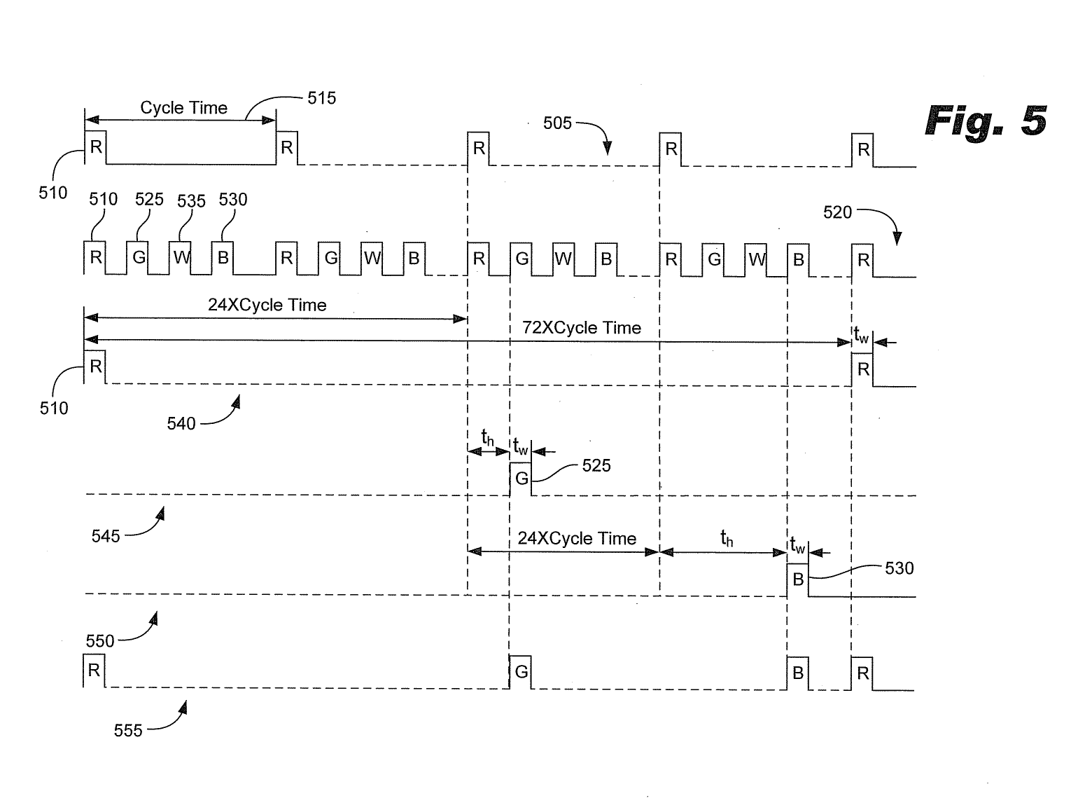

FIG. 4 illustrates a comparison of wide stripes (410) and fine stripes (420) in a saw-toothed rainbow lighting pattern (400) for rainbow 3D imaging. The pattern (400) may comprise wide stripes (410) and fine stripes (420) projected onto the specimen successively. 2D data or images under these structured light patterns may be captured by CCD cameras. Rainbow patterns may be reconstructed from 2D images. In order to three-dimensionally reconstruct an image from the two-dimensional data, two steps may be taken. First, a wide stripe (410) may be utilized to search for initial corresponding points on the images. Second, fine stripes (420) are employed to calculate more accurate correspondences. Wide stripes (410) provide much larger searching ranges along epipolar lines than fine stripes (420), as demonstrated by comparing a wide stripe measurement (430) to a fine stripe measurement (440). For example, a first point (450) may be taken by using the wide stripes (410). Then, the first point (450) may be used as the starting position in order find a more accurate second point (460) using the fine stripes (420).

In order to increase the speed of capturing a real-time 3D image, a rolling pattern projecting approach may be used. This involves projecting three wide-stripes (410) in the beginning of the sequence, followed by projecting the remainder of sequences using fine-stripes (420). The first frame of 3D data may be reconstructed as described before, using a wide-stripe (410) followed by a fine-stripe (420). Starting from the second frame of 3D data, the current approach only uses fine-stripes (420). Assuming that all the stripes are projected onto the specimen at a very high speed, the 3D data in a frame N is employed as starting points for a subsequent frame N+1 immediately following the frame N, because points taken from frame N will be relatively accurate with respect to the same points on the specimen in frame N+1. This method may be able to account for small movements in the specimen due to breathing or other factors, though the accuracy of this method of 3D reconstruction may diminish with larger movements. Depending on the capabilities, requirements, or uses of the system, variations of the rolling patterns projecting approach may be used, including more or fewer passes of the wide-stripe patterns, or utilizing either wide or fine stripes at different times. Other methods of lighting control and/or capturing for real-time 3D imaging may be used for reconstructing the images in conjunction with the time-domain and frequency-domain responses of the specimen.

FIG. 5 illustrates one example of a timing chart (500) for synchronizing color patterns from a projector in a spectrum source assembly (110) with capture cycles of a camera in a 3D imaging assembly (130). The timing may be important in order for the 3D camera to accurately capture the specimen responses to the correct color channels for the three-dimensional boundary reconstruction. A first waveform (505) is the basic signal generated from the color wheel of a projector. The basic signal is a red channel (510) sent at the beginning of every predetermined cycle time (515), though the basic signal may be any color channel, depending on the configuration of the projector. A second waveform (520) is the projector timing chart with red (R), green (G), blue (B), and white (W) channels (510, 525, 530, 535, respectively) of the projector. The red channel (510) in the second waveform (520) is matched with the red channel (510) of the first waveform (505). The green, blue, and white channels (525, 530, 535) may subsequently be arranged in any order following the red channel (510). If the basic signal of the first waveform (505) is any other color channel, the same color channel in the second waveform (520) will be matched with that of the first waveform (505), followed by the other channels. The white channel (535) is used to alter the brightness of the image, though no patterns are projected in the white channel (535). The second waveform (520) may comprise a plurality of white channels (535), depending on the desired brightness of the image. The second waveform (520) may be synchronized with the first waveform (505) such that the channel sequence of the second waveform (520) repeats at the same frequency of the basic signal in the first waveform (505).

Third, fourth, and fifth waveforms (540, 545, 550, respectively) are red, green, and blue channels (510, 525, 530, respectively) generated from a printed circuit board (PCB). The third, fourth, and fifth waveforms (540, 545, 550) are generated at different times and each may be generated after a predetermined number of cycle times (515). In the example of FIG. 5, these signals are generated every 24 cycle times (515), with the third waveform (540)—coming from the PCB board—generating a red channel (510) at the beginning of the sequence. A sixth waveform (555) is an output signal from the PCB board to trigger the camera such that it captures the correct red, green, and blue specimen responses to the spectrum source assembly (110). The sequence may start over after all of the responses have been adequately captured. This may be important to allow the system to recover, depending on the speed of the 3D camera and/or the projector, or other physical/mechanical limitations of the spectrum source assembly (110) and the 3D imaging assembly (130). In the current example, the sequence restarts after 72 cycle times (515) have passed since the beginning of the last occurrence of the sequence. Pulse widths and hold times of each signal may be calculated to allow for all of the signals to be cleanly sent and received without interfering with other signals.

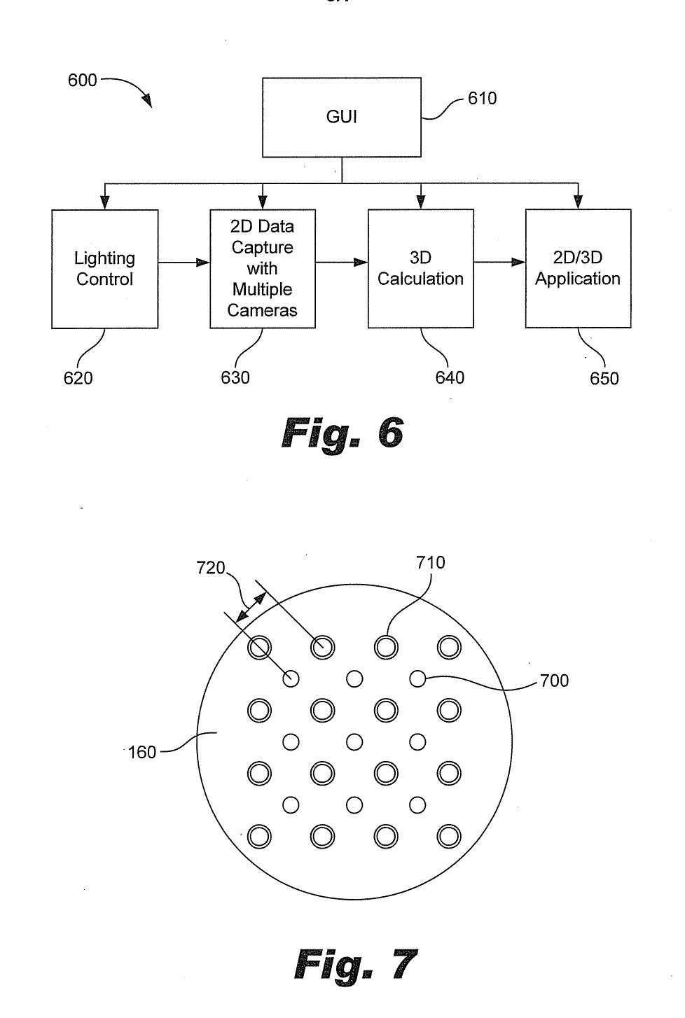

FIG. 6 is a block diagram (600) of one method of obtaining 3D data with a 3D imaging system through a graphical user interface (GUI) (610). A user may use the GUI (610) to control or direct one or more phases of the process. Phase-shifting structured light patterns are generated and projected onto the specimen for lighting control (620). If one or more color CCD cameras and structured (multi-rainbow) light are used to capture (630) a 2D color image, then the 3D surface data may be calculated (640) from the coded color image captured in one snap shot.

To get “pure rainbow color image,” to increase the accuracy of the 3D data, and at the same time avoid costly color CCD cameras, a method using black and white cameras and 3 phase-shifting light patterns (shifted 120 degrees from each other) may be used for the rainbow 3D camera. Three black and white images with phase-shifting structured light may be captured to represent the red, green, and blue components of the color images. The red, green, and blue components are then merged together to form a multi-rainbow color image and the 3D surface data is generated from this image. Finally, the obtained 2D/3D data can be used for various specific applications (650).

FIG. 7 is one embodiment of a plurality of optical source probes (700) and detection fibers (710) distributed on a supporting structure (160) where the specimen may be placed for imaging. The distribution of the probes (700) and fibers (710)—the number and locations of probes (700) and fibers (710) and the distance (720) between the probes (700) and detection fibers (710)—may be optimized such that the dynamic range of the system is maximized. In the illustrated embodiment, the larger, double circles represent the locations of the detection fibers (710), while the smaller circles represent the locations of the source probes (700). This configuration may allow for the source probes (700) to apply a relatively uniform illumination across the entire specimen. Also, the detection fibers (710) are positioned such that they may detect a wide range of responses of the specimen to the illumination. The source probes (700) and/or detection fibers (710) may be turned on individually or collectively to allow for various illumination patterns. Each source probe (700) may be configured to be capable of emitting different wavelengths of light. While the supporting structure (160) in the current embodiment is depicted as being circular for supporting a circular specimen, such as a breast phantom, the supporting structure may be any shape for supporting a specimen or test phantom. Accordingly, the source probes (700) and detection fibers (710) may be distributed in any manner sufficient to maximize the dynamic range of the system, depending on the shape of the desired supporting structure (160) and specimen.

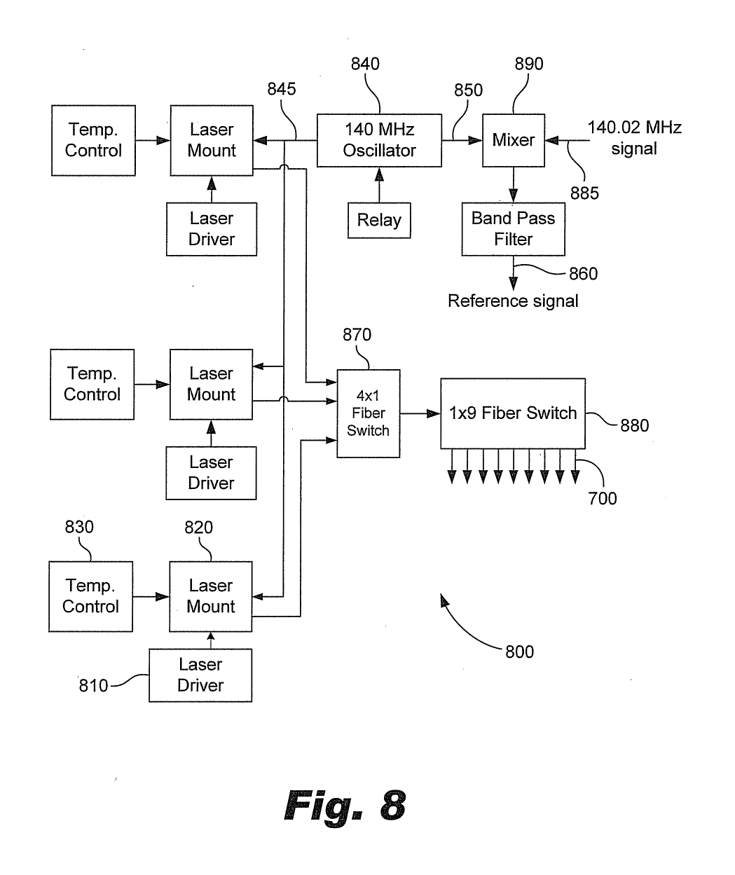

FIG. 8 shows a block diagram of one illustrative embodiment of a frequency-domain source module (800). The source module (800) described in the present embodiment provides 9 optical source probes (700) for emitting an illumination which can be distributed on the surface of a phantom or testing tissues. Each source probe (700) has an input which may be switched between 3 mixed near-infrared lasers of different wavelengths via optical switches, each laser being generated by an individual laser driver (810). The laser driver may be attached to a laser mount (820), and may comprise a temperature control (830) to maintain all of the laser drivers (810) operating at a constant temperature. All the lasers are modulated with a fixed oscillator (840). The frequency-domain source module (800) may use a plurality of wavelengths for determining various characteristics of the specimen. One wavelength which may be useful for determining oxygenation information is 690 nanometers (nm), due to its large absorption difference between deoxygenated hemoglobin and oxygenated hemoglobin, though any wavelength in the range for DOT imaging may be suitable for illuminating the specimen, generally from 650 nm to 850 nm. By using of a plurality of wavelengths, the hemoglobin oxygenation may be evaluated quantitatively.

In the current embodiment, the oscillator (840) is a 10-dBm (power ratio in decibels (dB) of the measured power referenced to one milliwatt (mW)) oscillator which generates 140 megahertz (MHz) sine waves for modulation. The oscillator (840) may have a main output (845) going through a directional coupler to a radio frequency (RF) amplifier (not shown), while an additional output (850) may be used to produce a reference signal (860) for a frequency-domain detection module. An attenuator may be used to reduce the power level of the output to the reference signal (860) if needed. The amplified 140 MHz signal is connected to each laser mount (820) comprising a laser diode driver board, where it is combined with direct current (DC) bias currents to feed laser diodes. Modulated optical outputs are conveyed to three input ports of a 4-by-1 optical switch (870). The remaining input port may be reserved for an additional wavelength. The output of the 4-by-1 optical switch (870) is directly hooked to the input of a 1-by-9 optical switch (880), whose output ports each correspond to one source probe (700).

The source module also includes a system to produce the reference signal (860). A 20 kilohertz (kHz) reference signal may be used to measure the phase difference between the source signal and the detected signal that passes through the phantom. The signal is produced by mixing the 140 MHz oscillator signal with a 140.02 MHz oscillator signal (885) from the detection module in a mixer (890), and then filtering out the 20 kHz signal. This process is only used in the frequency-domain system. Though the current embodiment has used a 140 MHz oscillator (840), the system may use any frequency or frequencies for illuminating the specimen.

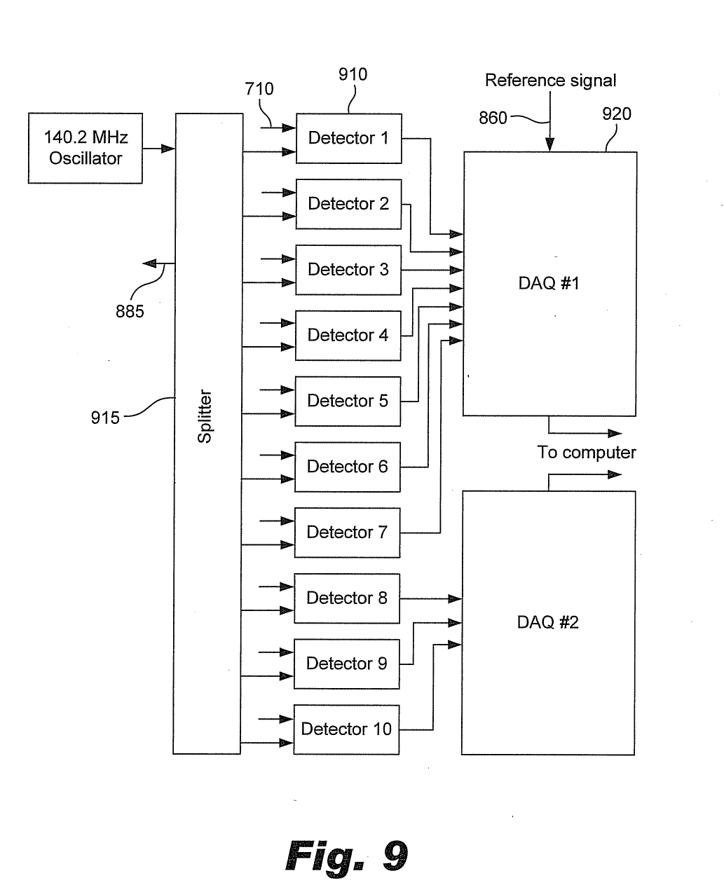

FIG. 9 shows a block diagram of one exemplary embodiment of a frequency-domain detection module (900). The frequency-domain detector module (900) is designed to detect the modulated laser signal that goes through the specimen or phantom and bounces back to the same side as the illumination source assembly. It consists of ten identical detectors (910), which are sealed in small boxes for radio frequency (RF) shielding. Consequently, in the illustrated embodiment there are ten detector fibers (710) in different positions on the specimen, as well as nine source probes in different positions.

The information that the detector module (900) produces can be used to produce tomography data. Modulated laser light at 140 MHz enters the phantom and part of it exits the specimen on the same side where it came from. The detector fibers (710) carry this light into photomultiplier (PMT) tubes inside the detectors (910). The PMT converts this light into electricity and produces a current proportional to the intensity of the light. This current is amplified and converted to a voltage signal by a transimpedance amplifier.

A signal processing PCB for PMT tubes in the detection module (900) may be very useful for data processing of the captured responses because of low signal to noise ratio requirements. Due to the very small variation in amplitude and phase of the detected signal between a healthy specimen and one with a tumor, the detectors need low signal to noise ratios in order to detect that small variation, as low as less than one percent. The transimpedance amplifier should be placed as close as possible to the output of the PMT to reduce the distance the very small signal has to travel. Proper grounding may also be very important in order to prevent excess noise in the detection module. Additionally, removing 90-degree turns and junctions on traces on the PCB allows for better transmission of high frequency signals.

This signal then goes into a mixer, which mixes it with a 140.02 MHz signal coming from a 1-by-12 splitter (915). The mixer outputs four different frequencies, the two original signals, 140 MHz and 140.02 MHz, as well as the sum and the difference of those signals, which are 280.02 MHz and 20 kHz. The 20 kHz signal is then filtered out using two consecutive active filters. These filters are made using operational amplifiers, and may filter and amplify at the same time. The 20 kHz signal is sent to a data acquisition (DAQ) card (920) so that it may be read by a computer. The output from the detectors (910) may be sent to more than one DAQ card (920), depending on the data capabilities of each DAQ card (920) and the number of detectors (910) used by the system. In the example of FIG. 9, the detection module (900) uses two DAQ cards (920) for handling the output of the ten detectors (910). This 20 kHz signal may be necessary due to limitations on the maximum sampling rate of the DAQ card (920). If the DAQ card (920) were able to support a sampling rate of 140 MHz, the 20 kHz would be unnecessary, and thus the 140 MHz signal would be able to be read directly by the computer. The reference signal (860) may be any frequency which the DAQ card (920) is capable of sampling. Other configurations of the detection module (900) which may not require the use of a DAQ card (920) may be used such that the original 140 MHz signal may be used by the computer directly.

The preceding description has been presented only to illustrate and describe embodiments and examples of the principles described. This description is not intended to be exhaustive or to limit these principles to any precise form disclosed. Many modifications and variations are possible in light of the above teaching.

What is claimed is:

1. A diffuse optical tomography imaging system for in vivo non-contact imaging, comprising:
an illumination source assembly for illuminating a specimen;
a time-domain sensor assembly for capturing a time-domain response of said specimen to illumination from said illumination source assembly;
a frequency-domain sensor assembly for capturing a frequency-domain response of said specimen to said illumination; and
a three-dimensional (3D) imaging assembly for outputting an electronic (3D) model of said specimen;
wherein said system combines said 3D model and tomography data generated from said time-domain response and frequency-domain response for said specimen.

2. The system of claim 1, wherein said illumination source assembly comprises a plurality of lasers, each outputting a beam having a different wavelength.

3. The system of claim 2, wherein said illumination source assembly comprises a plurality of optical source probes, each configured to output at least one of said lasers.

4. The system of claim 1, wherein said frequency-domain sensor assembly comprises a photomultiplier tube.

5. The system of claim 4, wherein said photomultiplier tube comprises a plurality of detection channels for detecting a response to the specimen at different positions.

6. The system of claim 5, wherein said detection channels comprise radio frequency shielding.

7. The system of claim 1, wherein said frequency-domain sensor assembly comprises a control voltage selector.

8. The system of claim 1, wherein said illumination source assembly is configured to switch between a continuous-wave beam and a frequency-modulated beam.

9. The system of claim 1, wherein said 3D imaging assembly comprises two separate real-time 3D cameras directed at different areas of said specimen.

10. The system of claim 1, wherein said system comprises two 3D imaging assemblies directed at opposite sides of said specimen.

11. The system of claim 1, wherein said system comprises a spectrum source assembly comprising a digital light processing (DLP) projector for projecting a multi-color spectrum on said specimen.

12. The system of claim 11, wherein said spectrum source assembly comprises a synchronizing trigger system for synchronizing said spectrum source assembly with said 3D imaging assembly.

13. The system of claim 1, wherein said system comprises a supporting structure for supporting said specimen.

14. The system of claim 1, further comprising a processor-based device configured to process data acquired by said time-domain sensor assembly, said frequency-domain sensor assembly, and said 3D imaging assembly.

15. The system of claim 14, wherein said processor-based device is further configured to control said illumination source assembly, said time-domain sensor assembly, said frequency-domain sensor assembly, and said 3D imaging assembly.

16. The system of claim 15, wherein said processor-based device comprises a user-interface program for allowing a user to control said system and view images produced from said tomography data and said 3D model.

17. A diffuse optical tomography imaging system for in vivo non-contact imaging, comprising:
means for illuminating a specimen;
means for sensing a time-domain response of said specimen to illumination;
means for sensing a frequency-domain response of said specimen to said illumination;
means for generating tomography data from said time-domain response and said frequency-domain response;
means for generating an electronic (3D) model of said specimen; and
means for combining said tomography data and said 3D model for said specimen.

18. A method for using a diffuse optical tomography imaging system for in vivo non-contact imaging, comprising:
illuminating a specimen;
capturing a time-domain response of said specimen to illumination with a time-domain sensor assembly, and a frequency-domain response of said specimen to said illumination with a frequency-domain sensor assembly;
generating tomography data from said time-domain response and said frequency-domain response;
generating an electronic (3D) model of said specimen; and
combining said tomography data and said 3D model for said specimen.15.

19. The method of claim 18, further comprising controlling said system with a graphical user interface program on a processor-based device connected to said system.

20. The method of claim 18, wherein generating said 3D model comprises using a rolling-patterns projection for capturing 3D data.
